SEQUENCE LISTING (4 sequences)

SEQ ID NO: 1 - DNA, 1188 bp, Xenopus laevis
atgcctaaaaagaagcctacgcccatacagctgaatcccaaccccgaagggactgctgtgaacgggacccctacagccgagacaaaccttgaagctctgcagaaaaagttggaagagcttgagctggatgagcagcagaggaagcgtctggaggcttttctcacccagaagcagaaagttggggaactgaaggatgacgactttgaaaaagtttcagagcttggagcaggcaacggaggagtggtgtttaaggtgtcccacaagccaaccagcttgattatggccaggaagttgattcatctggagattaagcctgcaatccgaaaccagattatccgagagttgcaggttctgcatgaatgtaactccccatacattgtggggttctatggggccttctacagtgatggagagatcagcatttgcatggaacacatggatggaggctcccttgatcaggttctgaagaaagctggcaaaatcccagaaaagattttgggaaaagtcagcattgcagtgataaaaggtctaacctacctgagagaaaagcataagataatgcacagagatgtgaaaccttctaacatcctggtcaactctagaggagagataaaactctgcgactttggggtcagcgggcaactcatagactccatggcaaattcctttgttgggacaagatcctatatgtcaccggagcgactacagggcactcattattctgtgcaatcagacatctggagcatggggctgtcgctggtggaaatggccattggaaggtatcccattccaccccctgatgccaaagagctggaacttatctttgggtgttctgtagaaagggatccagcgtcttctgaactggcacctcgcccccggccacccggacgtccaataagctcatacggtcctgatagtcgaccacccatggctatttttgaacttctggattatatcgtgaacgagccgcctccaaaattgcccagtggagtatttggagctgagttccaggactttgtgaataaatgtcttgtgaagaatccggcagagagagcagaccttaaacagctaatggttcacagcttcattaagcagtcagagttggaggaagtggattttgctggatggctctgttccactatgggccttaagcagcccagtaccccaacccatgccgccggagtgtga

SEQ ID NO: 2 - protein, 395 aa, Xenopus laevis
MPKKKPTPIQLNPNPEGTAVNGTPTAETNLEALQKKLEELELDEQQRKRLEAFLTQKQKVGELKDDDFEKVSELGAGNGGVVFKVSHKPTSLIMARKLIHLEIKPAIRNQIIRELQVLHECNSPYIVGFYGAFYSDGEISICMEHMDGGSLDQVLKKAGKIPEKILGKVSIAVIKGLTYLREKHKIMHRDVKPSNILVNSRGEIKLCDFGVSGQLIDSMANSFVGTRSYMSPERLQGTHYSVQSDIWSMGLSLVEMAIGRYPIPPPDAKELELIFGCSVERDPASSELAPRPRPPGRPISSYGPDSRPPMAIFELLDYIVNEPPPKLPSGVFGAEFQDFVNKCLVKNPAERADLKQLMVHSFIKQSELEEVDFAGWLCSTMGLKQPSTPTHAAGV

SEQ ID NO: 3 - DNA, 1188 bp, Artificial Sequence
  CAM cDNA
atgcctaaaaagaagcctacgcccatacaggcgaatcccaaccccgaagggactgctgtgaacgggacccctacagccgagacaaaccttgaagctctgcagaaaaaggcggaagagcttgagctggatgagcagcagaggaagcgtctggaggcttttctcacccagaagcagaaagttggggaactgaaggatgacgactttgaaaaagtttcagagcttggagcaggcaacggaggagtggtgtttaaggtgtcccacaagccaaccagcttgattatggccaggaagttgattcatctggagattaagcctgcaatccgaaaccagattatccgagagttgcaggttctgcatgaatgtaactccccatacattgtggggttctatggggccttctacagtgatggagagatcagcatttgcatggaacacatggatggaggctcccttgatcaggttctgaagaaagctggcaaaatcccagaaaagattttgggaaaagtcagcattgcagtgataaaaggtctaacctacctgagagaaaagcataagataatgcacagagatgtgaaaccttctaacatcctggtcaactctagaggagagataaaactctgcgactttggggtcagcgggcaactcatagacgacatggcaaatgagtttgttgggacaagatcctatatgtcaccggagcgactacagggcactcattattctgtgcaatcagacatctggagcatggggctgtcgctggtggaaatggccattggaaggtatcccattccaccccctgatgccaaagagctggaacttatctttgggtgttctgtagaaagggatccagcgtcttctgaactggcacctcgcccccggccacccggacgtccaataagctcatacggtcctgatagtcgaccacccatggctatttttgaacttctggattatatcgtgaacgagccgcctccaaaattgcccagtggagtatttggagctgagttccaggactttgtgaataaatgtcttgtgaagaatccggcagagagagcagaccttaaacagctaatggttcacagcttcattaagcagtcagagttggaggaagtggattttgctggatggctctgttccactatgggccttaagcagcccagtaccccaacccatgccgccggagtgtga

SEQ ID NO: 4 - protein, 395 aa, Artificial Sequence
  CAM amino acid
MPKKKPTPIQANPNPEGTAVNGTPTAETNLEALQKKAEELELDEQQRKRLEAFLTQKQKVGELKDDDFEKVSELGAGNGGVVFKVSHKPTSLIMARKLIHLEIKPAIRNQIIRELQVLHECNSPYIVGFYGAFYSDGEISICMEHMDGGSLDQVLKKAGKIPEKILGKVSIAVIKGLTYLREKHKIMHRDVKPSNILVNSRGEIKLCDFGVSGQLIDDMANEFVGTRSYMSPERLQGTHYSVQSDIWSMGLSLVEMAIGRYPIPPPDAKELELIFGCSVERDPASSELAPRPRPPGRPISSYGPDSRPPMAIFELLDYIVNEPPPKLPSGVFGAEFQDFVNKCLVKNPAERADLKQLMVHSFIKQSELEEVDFAGWLCSTMGLKQPSTPTHAAGV

(19) United States
(12) Patent Application Publication
(10) Pub. No.: US 2012/0269796 A1
(43) Pub. Date: Oct. 25, 2012

(54) Method for Enhancing Pancreatic Beta Cell Proliferation, Increasing Serum Insulin Concentration, Decreasing Blood Glucose Concentration And Treating And/Or Preventing Diabetes

(75) Inventors: Hideki KATAGIRI, Miyagi (JP); Yoshitomo OKA, Miyagi (JP); Junta IMAI, Miyagi (JP)

(73) Assignee: TOHOKU UNIVERSITY, Miyagi (JP)

(21) Appl. No.: 13/530,496

(22) Filed: Jun. 22, 2012

Related U.S. Application Data
(63) Continuation of application No. 12/165,859, filed on Jul. 1, 2008, now abandoned.
(60) Provisional application No. 61/076,802, filed on Jun. 30, 2008.

(51) Int. Cl. A61K 38/45; A61P 3/08; A61P 5/50; A61P 3/10; A61K 31/7088; A61P 1/18

(52) U.S. Cl. 424/94.5; 514/44 R

(57) ABSTRACT

By enhancing the function of ERK proteins in the liver, proliferation of pancreatic β cells is promoted, blood insulin concentration increased, blood glucose level decreased, and diabetes is prevented and/or treated. The methods for enhancing the function of ERK proteins in the liver are not particularly limited, and include various aspects such as enhancement of activity of MEK proteins, activation of endogenous MEK proteins, administration of an expression vector which expresses a gene encoding an active-form of MEK protein, and the like.

### FIELD OF THE INVENTION

The present invention relates to methods for promoting proliferation of pancreatic β cells, methods for increasing blood insulin concentration, methods for decreasing blood glucose levels, and methods for treating and/or preventing diabetes.

### BACKGROUND OF THE INVENTION

Type 1 diabetes is caused by insulin deficiency resulting from damaged lesion or loss of β cells which synthesize and secrete insulin in the islets of Langerhans of the pancreas. Patients with type 1 diabetes are thus pressed to undergo lifelong insulin treatment. However, as a matter of fact, even with frequent injections of insulin (e.g., 3 to 4 times daily), it is difficult to control blood glucose levels and therefore development of therapeutic agents and therapeutic methods for type 1 diabetes have been desired.

In recent years, therapeutic methods such as pancreas transplantation and pancreatic islet transplantation for type 1 diabetes patients have been under development (Ryan E A et al., Diabetes 54: 2060-2069, 2005). However, since pancreas transplantation and pancreatic islet transplantation involve the problems of rejections and an absolute shortage of donors, implementation of these transplantations is predicted to be difficult. For this reason, there has been a need for the development of agents and therapeutic methods for type 1 diabetes other than the pancreas transplantation or the pancreatic islet transplantation.

Furthermore, it was recently shown that the number of pancreatic β cell is reduced in patients with type 2 diabetes as well. With this as background, for not only type 1 diabetes but also for type 2 diabetes from which the majority of diabetics suffer, the development of a therapeutic method which brings about an increase in pancreatic β cells and insulin secretion is desired.

### SUMMARY OF THE INVENTION

The present invention relates to methods for promoting proliferation of pancreatic β cells, methods for increasing blood insulin concentration, methods for decreasing blood glucose levels, and methods for treating and preventing diabetes.

The inventors introduced a gene encoding an active-form of MEK protein into the liver of normal mice using an adenovirus and examined its effects. A glucose tolerance test at 3 days after the viral administration showed that the glucose-responsive blood insulin concentration increased and that the blood glucose levels during the glucose tolerance test decreased. Further, it was shown that the pancreatic insulin content at 16 days after the viral administration of the mice into which an active-form of MEK protein had been introduced increased to almost 3-fold, as compared with the pancreatic insulin content of the mice into which the LacZ gene had been introduced. Furthermore, in a histological examination, a significant increase in the ratio of BrdU-positive in pancreatic islets was noted in the mice into which the active-form of MEK protein had been introduced. Thus the inventors discovered that β cells proliferate in pancreatic islets of the mice into which an active-form of MEK protein has been introduced and accomplished the present invention.

In one embodiment of the present invention, a method for promoting proliferation of pancreatic β cells, a method for increasing blood insulin concentration, a method for decreasing blood glucose levels, a method for treating/preventing diabetes, and a method for stimulating the vagus nerve in the pancreas according to the present invention includes enhancement of the function of ERK proteins in the liver in a human or a non-human vertebrate. In any of the aforementioned methods, endogenous MEK proteins may be activated in the liver. Also, in any of the aforementioned methods, an active-form of MEK protein or a substance which causes expression of an active-form of ERK protein may be introduced into the liver. The substance which causes expression of an active-form of MEK protein may be an expression vector containing a gene which expresses the active-form of MEK protein in the liver. Types of diabetes to be prevented and/or treated include type 1 diabetes, type 2 diabetes, diabetes resulting from loss of pancreatic β cells, and diabetes resulting from pancreatic cell disorder.

The present invention has made it possible to newly provide a method for promoting proliferation of pancreatic β cells, a method for increasing blood insulin concentration, a method for decreasing blood glucose levels, and a method for treating and/or preventing diabetes.

### BRIEF DESCRIPTION OF THE DRAWINGS

FIG. 1 shows results of various measurements after an active-form of MEK gene was introduced into the liver of normal mice in one example according to the present invention. (a) shows the result of measurements of phosphorylation of ERK proteins; (b) shows the result of measurements of the blood glucose levels after a glucose tolerance test; (c) shows the result of a measurement of the serum insulin levels during the glucose tolerance test; and (d) shows the result of measurements of the pancreatic insulin content.

FIG. 2 shows results of histological analysis after an active-form of MEK gene was introduced to the liver of normal mice in one example according to the present invention. (a) shows the result of temporal measurements of the number of pancreatic islets; (b) shows the result of observation of BrdU-positive cells among pancreatic islet cells; (c) shows the result of measurements of the number of BrdU-positive cells among pancreatic islet cells; (d) shows the result of double staining of BrdU and insulin in pancreatic islet cells; and (e) shows the result of measurements of phosphorylation of ERK proteins in the isolated pancreatic islets.

FIG. 3 shows results of various measurements after an active-form of MEK gene was introduced to the liver of those mice who had undergone vagotomy in one example according to the present invention. (a) and (b) show the results of measurements of the blood glucose level and serum insulin level, respectively, during a glucose tolerance test; (c) shows the result of measurements of the pancreatic insulin content; and (d) shows the result of measurements of the number of BrdU-positive cells among pancreatic islet cells.

FIG. 4 shows results of measurements of activation of the ERK signal transduction pathway in the liver of type 2 diabetes model mice in one example according to the present invention. (a) shows the result of measurements of phosphorylation of ERK proteins in the liver of type 2 diabetes model mice; (b) shows the result of measurements of phosphorylation of ERK proteins in the liver of type 2 diabetes model mice, obtained when the dominant-negative mutant MEK1 gene was introduced; and (c) shows the result of measurements of the pancreatic insulin content of the type 2 diabetes model mice obtained when the dominant-negative mutant MEK1 was introduced.

FIG. 5 shows results of various measurements after an active-form of MEK gene was introduced into the liver of type 1 diabetes model mice (STZ mice) in one example according to the present invention. (a) shows the result of measurements of fasting blood glucose levels; (b) shows the result of measurements of the number of BrdU-positive cells among pancreatic islet cells; and (c) shows the result of measurements of the pancreatic insulin content.

FIG. 6 shows results of various measurements after an active-form of MEK gene was introduced into the liver of type 1 diabetes model mice (AKITA mice) in one example according to the present invention. (a) shows the result of measurements of fasting blood glucose level; (b) shows the result of measurements of the number of BrdU-positive cells among pancreatic islet cells; and (c) shows the result of measurements of the pancreatic insulin content.

FIG. 7 shows the results of various measurements after an active-form of MEK gene was introduced to the liver of type 2 diabetes model mice (db/db ksj mice) in one example according to the present invention. (a) shows the result of measurements of fasting blood glucose levels; (b) shows the result of measurements of the number of BrdU-positive cells among pancreatic islet cells; and (c) shows the result of measurements of the pancreatic insulin content.

### DETAILED DESCRIPTION OF THE PREFERRED EMBODIMENTS

Embodiments of the present invention accomplished based on the above-described findings are hereinafter described in detail by giving Examples, though the present invention is not limited to these Examples.

Unless otherwise explained, methods described in standard sets of protocols such as J. Sambrook and E. F. Fritsch & T. Maniatis (Ed.), “Molecular Cloning, a Laboratory Manual (3rd edition), Cold Spring Harbor Press and Cold Spring Harbor, N.Y. (1989); and F. M. Ausubel, R. Brent, R. E. Kingston, D. D. Moore, J. G. Seidman, J. A. Smith, and K. Struhl (Ed.), “Current Protocols in Molecular Biology,” John Wiley & Sons Ltd., or alternatively, their modified/changed methods are used. When using commercial reagent kits and measuring apparatus, unless otherwise explained, protocols attached to them are used.

The object, characteristics, and advantages of the present invention as well as the idea thereof will be apparent to those skilled in the art from the descriptions given herein. It should be understood that the embodiments and specific examples of the invention described herein below are to be taken as preferred examples of the present invention. These descriptions are only for illustrative and explanatory purposes and are not intended to limit the invention to these embodiments or examples. It is further apparent to those skilled in the art that various changes and modifications may be made based on the descriptions given herein within the intent and scope of the present invention disclosed herein.

===Pharmacological Effects===

As shown in the following examples, the inventors incorporated a gene encoding an active-form of MEK protein into an adenovirus which is capable of efficient gene transfer to the liver and introduced the active-form of MEK protein into the liver of normal mice (C57Bl/6N mice) using this adenovirus, thereby made it possible to promote proliferation of pancreatic β cells for increasing the pancreatic insulin content to decrease the blood glucose levels.

MEK protein, a component of the ERK signal transduction pathway, also called ERK kinase, has the function of phosphorylating and activating ERK proteins in cells. Since the MEK protein functions via ERK proteins, the substance which enhances the function of ERK proteins in the liver can be used to promote proliferation of pancreatic β cells, to increase blood insulin concentration, to decrease blood glucose levels, and to prevent or treat diabetes. In addition, the ERK signal transduction pathway controls fundamental cellular functions. Since such functions are conserved in a wide variety of animal species, the animal species from which the components of the ERK signal transduction pathway such as MEK protein and ERK proteins are derived are not particularly limited.

The details are described as follows.

(1) Method for Promoting Proliferation of Pancreatic β Cells

The inventors introduced a gene encoding an active-form of MEK into the liver of normal mice and then measured the number of pancreatic β cells as well as the area of the pancreatic islets so that proliferation of β cells was found to have been promoted in the pancreatic islets of these mice. It is therefore useful as a method for promoting proliferation of pancreatic β cells to enhance the function of ERK proteins in the liver.

(2) Method for Increasing Blood Insulin Concentration

The inventors also introduced a gene encoding an active-form of MEK into the liver of normal mice and then measured the pancreatic insulin content so that the pancreatic insulin content was found to have increased. Since the insulin synthesized in the pancreas will be secreted into the blood, an increase in the pancreatic insulin content leads to an increase in blood insulin concentration. In fact, it was shown in a glucose tolerance test that glucose-responsive insulin secretion increased. Therefore, it is useful as a method for increasing blood insulin concentration to enhance the function of ERK proteins in the liver.

(3) Method for Decreasing Blood Glucose Levels

Further, the inventors introduced a gene encoding an active-form of MEK into the livers of normal mice and then measured fasting blood glucose levels so that fasting blood glucose levels were found to have decreased to normal levels. Moreover, the inventors introduced a gene encoding an active-form of MEK into the livers of normal mice, using the same methods as described above, and then performed a glucose tolerance test and measured the blood glucose levels so that the blood glucose level 2 hours after the glucose loading was found to have decreased to normal levels. The “normal levels” herein refers to the blood glucose levels of a healthy mouse without a disease or the like. Therefore, it is useful as a method for decreasing the blood glucose level to enhance the function of ERK proteins in the liver.

(4) Method for Preventing/Treating Diabetes

The inventors introduced a gene encoding an active-form of MEK into the livers of type 1 diabetes model mice (STZ mice: diabetes model mice which develop hyperglycemia due to the lesion of pancreatic β cells), diabetes model mice which develop progressive loss of pancreatic β cells (AKITA mice, diabetes model mice which develop hyperglycemia due to apoptosis of pancreatic β cells resulting from endoplasmic reticulum stress), and model mice which develop type 2 diabetes as a result of obesity (C57 BL/KSJ-db/db mouse: diabetes model mice which develop hereditary obesity as a result of leptin receptor mutation and become insulin resistant), and then measured the pancreatic insulin contents and fasting blood glucose levels, so that it was found that pancreatic insulin contents have increased and fasting blood glucose levels have decreased to the normal levels. Therefore, the enhancement of the function of ERK proteins according to the present invention is useful as a method for preventing/treating diabetes.

Type 1 diabetes generally develops insulin deficiency due to destructive lesion of pancreatic β cells and the patients suffer from absolute deficit of insulin in many cases. On the other hand, type 2 diabetes is the most common type from which more than 95% of diabetic patients suffer, and it is suggested that decrease in secretion of insulin and sensitivity to insulin are involved in the development of this type of diabetes. In recent years, however, it has been clarified that, in development of type 2 diabetes as well, insulin deficiency occurs due to β cell disorder in close association with the cellular stress induced by insulin resistance. Therefore, the enhancement of the function of ERK proteins in the liver according to the present invention is useful not only as a method for preventing and treating type 1 diabetes due to the lesion or loss of pancreatic β cells but also as a method for preventing and treating type 2 diabetes which leads to a secondary pancreatic β cell disorder (for example, diabetes due to obesity or insulin resistance). The pancreatic β cell disorders include pancreatic β cell dysfunctions such as insulin secretion insufficiency, abnormal pancreatic β cell differentiation, abnormal pancreatic β cell proliferation, reductions of pancreatic β cell mass such as vulnerability of existing pancreatic β cells and disorders in the proliferation of existing pancreatic β cells, pancreatic β cell fatigue, etc.

In addition, the method for preventing/treating diabetes according to the present invention is also useful for secondary diabetes resulting from acute pancreatitis, chronic pancreatitis, necrotic pancreatitis, pancreatic cancer, congenital anomaly, autoimmune disease, medication side effects, environmental factors, etc.

Moreover, for prevention of the onset of diabetes or treatment of diabetes, the administration of the agent according to the present invention may be combined with an insulin therapy, etc. in the case of type 1 diabetes, and with an administration of an inhibitor of postprandial hyperglycemia, such as an α-glucosidase inhibitor (α-GI) and a rapid-acting insulin secretion promoter, a diet therapy, an exercise therapy, etc. in the case of type 2 diabetes.

===Method for Enhancing the Function of ERK Proteins===

The method for enhancing the function of ERK proteins in cells is not particularly limited as long as the ERK signal transduction pathway can be activated; endogenous ERK proteins within cells maybe activated, or an active-form of ERK protein or a substance which causes expression of an active-form of ERK protein may be introduced into the cells. Thus, the substance for enhancing the function of ERK proteins is not particularly limited as long as it is capable of activating the ERK signal transduction pathway.

(1) Method for Introducing an Active-Form of ERK Protein or a Substance which Causes Expression of an Active-Form of ERK Protein

The method for introducing an active-form of ERK protein into cells is not particularly limited. A fusion protein containing TAT or VP22 may be used as a protein transduction domain (PTD) fusion protein. An active-form of MEK protein synthesized in vitro may be introduced into cells, for example, by a protein delivery reagent, such as BioPorter™ or Chariot™, or by injection and the like.

The method for introducing a substance which causes expression of an active-form of ERK protein is not particularly limited. The calcium phosphate transfection method, lipofection method, electroporation method, microinjection method, viral infection method, or various other drug delivery systems (DDS) may be performed.

### (2) Method for Activating Endogenous ERK Proteins

To activate endogenous ERK proteins in cells, an agent (e.g., TPA) which activates ERK proteins may be used, or the upstream of the ERK signal transduction pathway may be activated.

To activate the upstream of the ERK signal transduction pathway in cells, for example, the function of MEK protein may be enhanced. As the method for the enhancement, endogenous ERK proteins may be activated; alternatively, an active-form of ERK protein or a substance which causes expression of an active-form of ERK protein may be introduced into the cells.

To activate endogenous MEK protein in cells, for example, the upstream of MEK proteins may be activated in the ERK signal transduction pathway. The method for this activation is not particularly limited, and for example, the RAS protein or MEK kinases such as the Raf protein or the MEKK-1 protein may be activated. The method for the activation of these is not particularly limited, and an activating factor for the ERK signal transduction system such as EGF may be administered, or an active-form of Raf protein or a substance which causes expression of an active-form of Raf protein may be introduced into the cells. The method for that is not particularly limited, and an active-form of Raf protein may be injected into the cells, or an expression vector encoding an active-form of Raf protein may be introduced into the cells by microinjection, lipofection, viral infection, etc.

To introduce an active-form of MEK protein or a substance which causes expression of an active-form of MEK protein, one of the methods for activating ERK proteins as described in (1) may be applied to an active-form of MEK protein.

(3) Active-Form of Proteins and Substances which Causes Expression of an Active-Form of Protein

An active-form of Raf protein, an active-form of MEK protein, or an active-form of ERK protein can be prepared by substituting in advance an acidic amino acid for the amino acid which would be phosphorylated when each protein is activated. Specifically, each protein can be activated by substituting an aspartic acid or a glutamic acid, for example, for serine at position 338 in the case of the Raf protein, for serine at position 218 as well as serine at position 222 in the case of an MEK protein, and for threonine at position 183 as well as tyrosine at position 185 in the case of an ERK protein.

The substance which causes expression of an active-form of protein is not particularly limited, as long as the substance causes expression of an active-form of protein in cells, and examples include an mRNA encoding an active-form of protein and an expression vector which has a gene encoding an active-form of protein.

The expression vector which expresses a gene encoding an active-form of protein maybe any type of vector, including a plasmid vector or a viral vector, as long as it contains a promoter which is operable in a host cell into which it is introduced. In addition, gene transfer may be implemented either by transient expression, in which the expression vector is extrachromosomally localized after the expression vector has been introduced, or by permanent expression, in which the expression vector is incorporated into a chromosome.

===Method for Preparing the Above-Mentioned Agents===

For a pharmacologically acceptable carrier to be used in the agents which enhance the function of the ERK proteins, one or more kinds of various conventional organic or inorganic carrier substances may be used as materials for preparation. For example, an excipient, a lubricant, a binder, and a disintegrator may be contained in a solid preparation, and a solvent a solubilizing agent, a suspending agent, a tonicity adjusting agent, a buffer, a soothing agent, etc. may be contained in a liquid preparation. In addition, a suitable amount of additives such as an ordinary preservative, an antioxidant, a colorant, a sweetening agent, a sorbent, a wetting agent, etc. can also be contained, if necessary. As for dosage forms, oral formulations include tablets, capsules, granule, powder, subtle granule, syrups, sustained-release tablets/capsules/granules, cachet, chewable tablets, or drops, and injections include liquid injections, emulsified injections, solid injections, etc.

Moreover, a carrier may be included, if necessary, depending on the dosage regimen. Lipid molecules in the case of lipofection method or the like is one such example.

===Mode of Administration of the Above-Mentioned Agents in Individual Animals===

To administer the agents according to the present invention to individual animals, the mode of administration to a cell as mentioned above can be applied to an individual animal.

The dosage of the agents according to the present invention varies depending on the age, body weight, indication, route of administration/intake, and is not particularly limited as long as it enables the agents to exert their actions with acceptable side effects.

The route of administration of the agents according to the present invention is not particularly limited as long as it enables enhancement of the function of ERK proteins in the liver.

In an exemplary situation in which the agent according to the present invention is to be administered, fasting blood glucose levels or the blood glucose level of 2 hours after a glucose tolerance test is measured in a human or a non-human vertebrate. If the blood glucose level is abnormally high, the agent which enhances the function of ERK proteins according to the present invention is administered to the subject.

It should be noted that in the cases of a patient with borderline diabetes, a patient having typical diabetic symptoms (thirst, excessive drinking, polyuria, and weight loss), a patient whose Hb_{Alc} is judged to be higher (for example, 6.5% or more) than that of a normal person, and a patient with complications, such as diabetic retinopathy, the agents according to the present invention may be used to prevent the development of diabetes or to treat diabetes.

===Method for Stimulating the Pancreatic Vagus Nerve===

When the vagus nerve controlling the pancreas in normal mice is cut, those symptoms (proliferation of pancreatic β cells, increased blood insulin concentration, and decreased blood glucose levels) which have been noted in normal mice into which a gene encoding an active-form of MEK protein has been introduced are not almost recognized. Therefore, activation of ERK proteins in the liver leads to stimulation of the vagus nerve controlling the pancreas. For this reason, to stimulate the vagus nerve controlling the pancreas in a human or a non-human vertebrate, the function of ERK proteins in the liver may be enhanced. Here, in the case of a human, “the vagus nerve controlling the pancreas” is the Xth cranial nerve arising from the brain neurons in the medulla oblongata. The method for “enhancing the function of ERK proteins in the liver” is the same as described above.

### EXAMPLES

Hereinafter, the embodiments described above will be further specifically explained using examples, which are provided solely for purposes of illustration and are not to be construed to limit the present invention to these examples.

### Example 1

===Effect of Introduction of an Active-Form of MEK Gene into the Liver of Normal Mice===

In this example, a gene encoding an active-form of MEK protein was introduced into the liver of normal mice (wild-type mice), and blood glucose levels and serum insulin levels as well as the number of pancreatic cells and number of pancreatic islets were measured in the mice.

(1) Test Animals

In this example, 8-week-old C57Bl/6N male mice (Kyudo Co., Ltd.) were used.

(2) Generation of Genetically-Engineered Adenoviruses

Using the MEK1 protein of Xenopus laevis (the nucleotide sequence of MEK1 cDNA and the amino acid sequence of MEK1 are shown in SEQ ID NO: 1 and SEQ ID NO: 2, respectively) (Fukuda M, Gotoh I, Adachi M, Gotoh Y, Nishida E: A novel regulatory mechanism in the mitogen-activated protein (MAP) kinase cascade. Role of nuclear export signal of MAP kinase kinase. J Biol Chem 272: 32642-32648, 1997), a gene (hereinafter described as the CAM gene) (the nucleotide sequence of CAM cDNA and the amino acid sequence of CAM are shown in SEQ ID NO: 3 and SEQ ID NO: 4, respectively) encoding the active-form of MEK1 protein in which serine at position 218 has been substituted by aspartic acid; serine at position 222 by glutamic acid; and leucines at positions 11 and 37 by alanine was constructed. Specifically, using a cDNA encoding the MEK1 protein of Xenopus laevis as a template, PCR reaction was performed with primers containing base substitutions for introducing the above-mentioned amino acid mutations to generate double-stranded DNA having the mutated sequence. By substituting part of wild-type cDNA with the mutated DNA fragment by digestion with restriction enzymes and ligation, a full-length CAM gene was constructed. Further, the CAM gene was incorporated into a cosmid in which the E1 region of adenoviral genome is substituted so that it can be expressed under the control of CAG promoter. After digestion with the restriction enzyme EcoT22I, the cosmid was transfected into HEK293 cells with DNA-terminal protein complexes (the COS-TPC method) (Miyake S, Makimura M, Kanegae Y, Harada S, Sato Y, Takamori K, Tokuda C, Saito I. Efficient generation of recombinant adenoviruses using adenovirus DNA-terminal protein complex and a cosmid bearing the full-length virus genome. Proc Natl Acad Sci USA, 93: 1320-1324, 1996) to construct an adenoviral vector expressing the CAM gene (hereafter described as CAM-Ad). As a control, a genetically-modified adenoviral vector constructed by incorporating β-galactosidase gene (hereafter described as LacZ-Ad) was used.

(3) Introduction of the CAM Gene into the Liver of Normal Mice

(i) Activation of the ERK Pathway

The liver of normal mice were infected with either of the adenoviruses (CAM-Ad or lacZ-Ad) (1.5×10⁸ PFU/body) by infusing it into the caudal vein of normal mice (C57Bl/6N). At 3 days after the viral administration, the degree of phosphorylation of ERK in the liver was measured by the Western blotting method. The following procedure was used for the measurement.

First, the liver of each mouse was excised, placed in the buffer (100 mM Tris pH 8.5, 250 mM NaCl, 1% BP-40, 1 mM EDTA, 40 mM Glycerol 2-phosphate, 50 mM NaF, 1 mM Na₃VO₄, 350 μg/ml phenylmethane sulfonyl fluoride, 2 μg/ml Aprotinin, 2 μg/ml Leupeptin) and homogenized on ice. The homogenized tissue was centrifuged at 14,000×g at 4° C. for 10 min, and then the supernatant containing protein extract (180 μg total protein) was boiled in Laemmli buffer containing 10 mM dithiothreitol. Next, the amount of the protein was quantified using Protein assay kit (Bio-Rad, Heracules, Calif.), and 15 μg of protein per sample was subjected to SDS-PAGE. Then, the protein was blotted onto the membrane using the conventional methods and the phosphorylation of ERK was detected using ECL plus a Western Blotting Detection System Kit (Amersham, Buckinghamshire, UK) with anti-phosphorylated ERK antibody (#4376, Cell Signaling Technology, Danvers, Mass.). To detect ERK protein expression level, anti-ERK antibody (#9102, Cell Signaling Technology) was used.

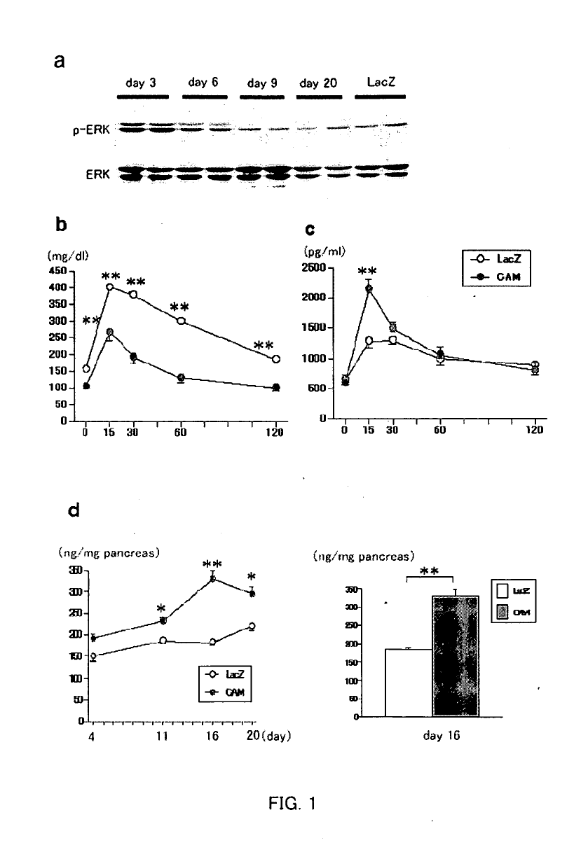

As a result, a significant increase in phosphorylation was found in the liver of the CAM gene-transferred mice (hereafter described as the CAM mice), compared with that of the LacZ gene-transferred mice (hereafter described as the LacZ mice) (FIG. 1a), indicating that the livers of the mice were infected with CAM-Ad.

(ii) Glucose Tolerance Test

A glucose tolerance test was performed on the CAM mice and LacZ mice generated in the same manner as in (i). First, at 3 days after the viral administration, a glucose load of 2 g/kg body weight was intraperitoneally administered. Blood samples were obtained from the caudal vein before glucose loading (0 min), at 15 min, 30 min, 60 min, and 120 min after loading, and the blood glucose levels and the serum insulin levels were measured. The insulin levels were measured with the High Sensitivity Mouse Insulin Assay Kit or the Ultra-High Sensitivity Mouse Insulin Assay Kit (Otsuka Pharmaceutical, Inc.).

As shown in FIG. 1b, both fasting blood glucose and post-load blood glucose levels were significantly decreased in the CAM mice, compared with those in the LacZ mice. Meanwhile, as shown in FIG. 1c, the serum insulin levels in the CAM mice significantly increased at min after glucose loading. It was thus shown that glucose-responsive insulin secretion increased in the CAM mice, resulting in decreased blood glucose levels during the glucose tolerance test.

Further, the pancreatic insulin content at 4, 11, 16, and 20 days after the viral administration was measured as follows. First, about two-thirds of the pancreas from its tail was removed from each mouse, homogenized in acid/ethanol, and stored in a −20° C. freezer. The next day and the day after, samples were ultrasonicated for 5 min and then centrifuged at 10, 000 rpm for 10 min. The supernatant was removed, and insulin levels were measured with the above-mentioned kit. The resultant values were corrected with the weight of the harvested pancreas and taken as pancreatic insulin concentration.

As shown in FIG. 1d, after the viral infection, the pancreatic insulin content gradually increased to almost twice the content in the LacZ mice at 16 days after the viral administration. These findings indicate that by expressing active-form of MEK in the liver, fasting blood glucose and post-load blood glucose levels decreased and glucose-responsive insulin secretion increased.

(iii) Histological Examination

Further, the pancreas after viral administration was histologically observed. Specific procedures were as follows. First, whole pancreases were removed from mice at 3, 9, 15, and 21 days after the viral administration. 3 μm sections of the pancreas were made at 500 μm interval. Insulin staining was performed on these sections and the total number of the pancreatic islets on the sections was counted. The counts of the pancreatic islets were standardized by being divided by the number of the histological sections, and the numbers of the pancreatic islets per unit volume were compared.

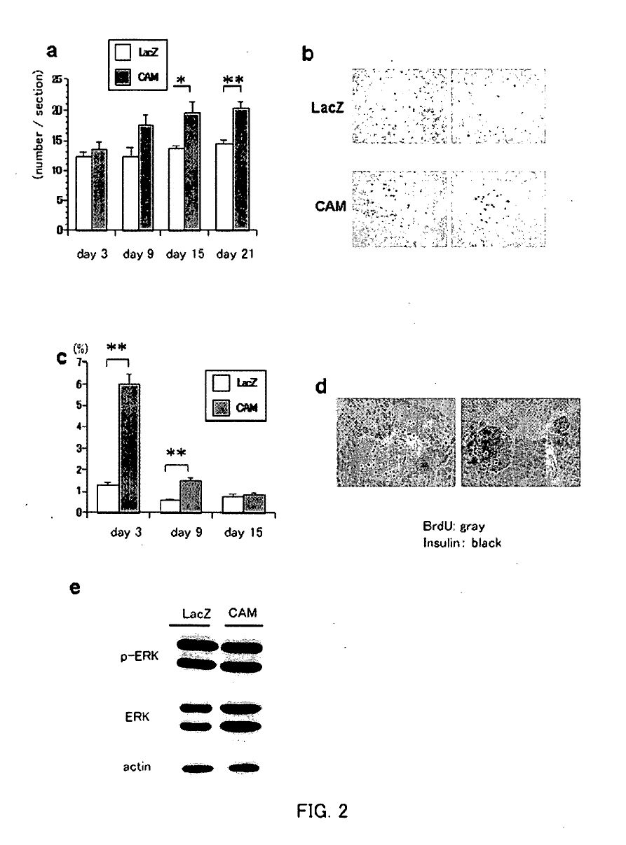

As shown in FIG. 2a, in this histological examination, the number of the pancreatic islets increased over time and a significant increase was observed in the CAM mice on and after 15 days after the viral administration compared with those of the LacZ mice.

Next, cell proliferation was examined by BrdU staining using a BrdU assay kit (BD bioscience, San Jose, Calif.). Specifically, 1 ml of BrdU (1 mg/ml) was intraperitoneally administered at 3, 9, 15, and 21 days after the viral administration. Twenty-four hours later, whole pancreas were removed and the specimen was cut into serial sections as described above, which were subjected to the following immunostaining of BrdU. First, The sections were antigen-retrieved by heating in the mixed solution of solution 1 and solution 2 included in the kit at 89° C. in an autoclave. Then, a 1:10 diluted solution of the biotinylated anti-BrdU antibody included in the kit was dribbled onto the sections, followed by an antigen-antibody reaction at room temperature for 1 h. Further, streptavidin-HRP included in the kit was added and reacted at room temperature for 30 min. Then, color was developed by reacting with 3,3′-diaminobenzidine (DAB) chromogen solution for 5 min.

As shown in FIG. 2b, a significant increase in the ratio of BrdU-positive cells among pancreatic islet cells of the CAM mice was observed at 3 days after the viral administration. The significant increase in the BrdU-positive cell ratio was sustained up to 9 days after the viral administration, but no difference was recognized between the CAM mice and the LacZ mice at 15 days after the viral administration (FIG. 2c). This indicated that cells with increased proliferation existed in the pancreatic islets of the CAM mice immediately after the virus administration.

Further, to examine the cell type(s) exhibiting the increased proliferation, double staining for BrdU and insulin was performed using pancreatic sections obtained from the CAM mice at 3 days after the viral administration. For the double staining, BrdU staining was performed with the first color reaction using DAB solution as described above (shown in gray in FIG. 2d, though actual color was brown) and then the second color reaction was performed using an aminoethyl carbazole (AEC) substrate kit (Nichirei Corporation) on the sections on which the primary antibody reaction had been performed with anti-insulin antibody (Sigma, St. Louis, Mo.) at room temperature for 1 h, followed by the secondary antibody reaction performed with peroxidase-labeled secondary antibody (Nichirei) at room temperature for 1 h (shown in black in FIG. 2d, though actual color was red).

As a result, as shown in FIG. 2d, it was revealed that 97.6% of the cells having a gray signal had a black signal, i.e., 97.6% of the BrdU-positive cells in the pancreatic islets were also positive to insulin.

Since the ERK pathway has an important function on cell proliferation (Roux P P, Blenis J: ERK and p38 MAPK-activated protein kinases: a family of protein kinases with diverse biological functions. Microbiol Mol Biol Rev 68: 320-344, 2004), a possibility was that CAM Ad might have directly acted on the β cells to promote their proliferation.

Thus, the pancreatic islets which had been isolated from the LacZ and CAM mice at 3 days after the viral administration were subjected to the Western blotting method using an anti-phospho-ERK antibody in order to examine the degree of phosphorylation of ERK.

First, the pancreas was excised from respective mice and homogenized in a buffer (100 mM Tris pH 8.5, 250 mM NaCl, 1% BP-40, 1 mM EDTA, 40 mM Glycerol 2-phosphate, 50 mM NaF, 1 mM Na₃VO₄, 350 μg/ml phenylmethane sulfonyl fluoride, 2 μg/ml Aprotinin, 2 μg/ml Leupeptin). The homogenized tissue was centrifuged at 14,000×g at 4° C. for 10 min, and then the supernatant containing protein extract (180 μg total protein) was boiled in Laemmli buffer containing 10 mM dithiothreitol. After SDS-PAGE was performed, the proteins were transferred to the membrane and phosphorylated ERK was detected with the anti-phospho-ERK antibody (#4376, Cell Signaling Technology, Danvers, Mass.). The detection for obtaining the result was performed using ECL plus a Western Blotting Detection System Kit (Amersham Buckinghamshire, UK). Western blotting using an anti-ERK antibody (#9102, Cell Signaling Technology) confirmed no change in the expression level of ERK proteins.

As a result, as shown in FIG. 2e, no clear difference in the phosphorylation level of ERK was observed between the two groups. It was therefore concluded that the cell proliferation in the pancreas was not due to the action of CAM Ad on the pancreas.

These findings indicated that the proliferation of the β cells in the pancreatic islets of the CAM mice was enhanced by a functional mechanism other than the direct effect of promoting proliferation resulting from the infection of CAM Ad to the pancreas so that the number of pancreatic islets increased, leading to an increase in pancreatic insulin content.

### Example 2

===Involvement of the Vagus Nerve Controlling the Pancreas in the Proliferation of Pancreatic β Cells by Introduction of the CAM Gene into the Liver===

It is known that the pancreas is innervated by each of posterior and anterior esophageal vagal trunks running along the ventral and dorsal esophagus. Thus, mice were laparotomized via midline incision, esophagogastric junction was exposed, and the anterior vagal trunk running along the ventral esophagus was cut near the esophagogastric junction. Subsequently, intraperitoneal organs such as the enteric canals, stomach, spleen, etc. were moved to the right-hand side to expose the area around the celiac artery branching from the abdominal aorta. The celiac branch of the vagus nerve, which branches off from the posterior vagal trunk running along the dorsal esophagus and which lies along the celiac artery, was cut at the site nearest possible to the pancreas (vagotomy (VG)). Mice were subjected to viral injection after 1 week of postoperative convalescence. The results of the analysis obtained after the viral injection was compared with those of a group in which mice had undergone a simple operation consisting of laparotomy followed by exposure of the esophagogastric junction and the celiac artery (sham-operation (SO) group).

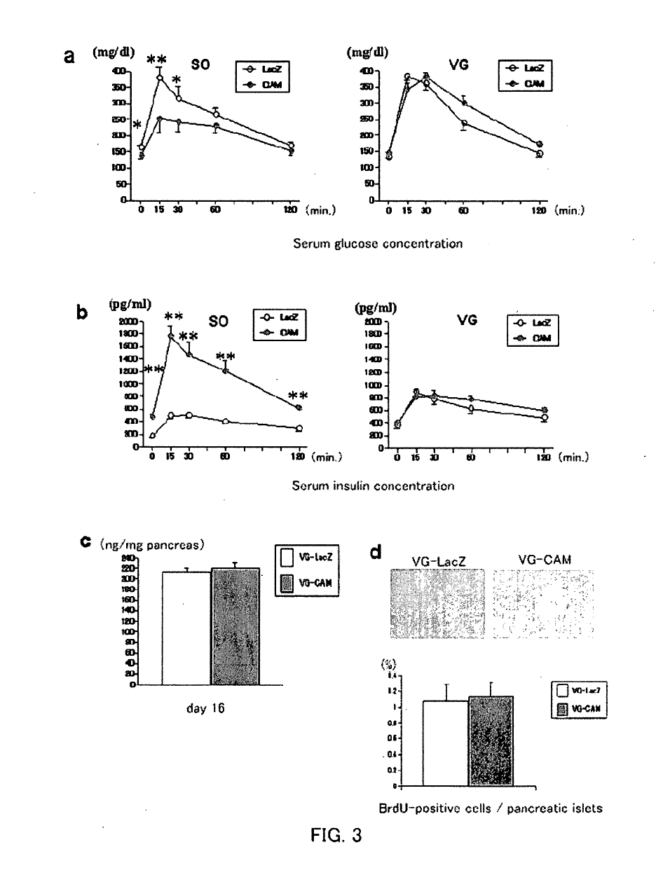

In the mice which underwent SO followed by CAM viral injection (SO-CAM mice), the decrease of blood glucose levels during the glucose tolerance test at 3 days after the viral administration, the increase of glucose-responsive insulin secretion, and the increase of pancreatic insulin content (on day 16) were observed, whereas all of them were abolished in the mice which underwent VG followed by CAM viral injection (VG-CAM mice) almost completely (FIG. 3a, b, c). Also in the VG-CAM mice, the increase in the BrdU-positive cells in pancreatic islets at 3 days after the viral administration, which occurred in the SO-CAM mice, was not observed (FIG. 3d).

These findings indicate that the increase of glucose-responsive insulin secretion by the CAM gene transfer to the liver and the increase of pancreatic insulin content by the proliferation of pancreatic β cells occur via the vagus nerve controlling the pancreas, as well as that these effects are not caused by the viral infection of CAM into the pancreas.

### Example 3

===Involvement of the Hepatic ERK Pathway in Pancreatic Islet Hypertrophy of Insulin-Resistance Model Mice===

In this example, involvement of the hepatic ERK pathway in pancreatic islet hypertrophy of insulin-resistance model mice was investigated.

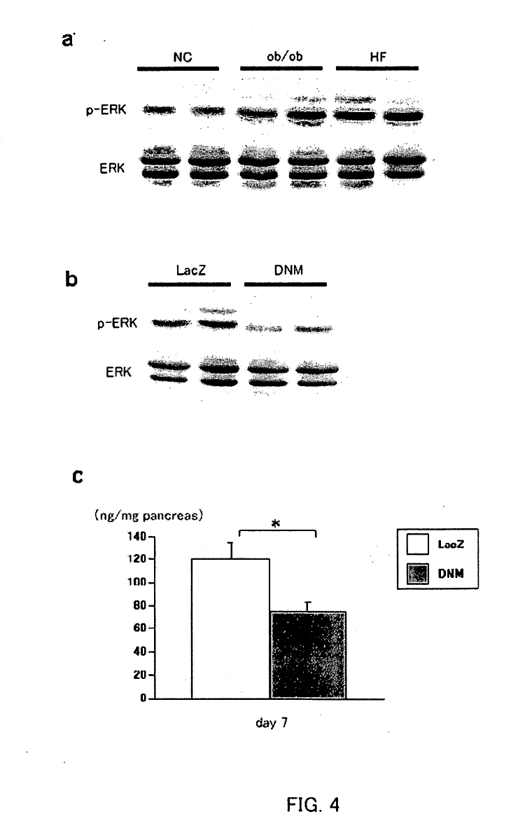

It has previously been reported that the phosphorylation of ERK increased in the liver of ob/ob mice (Yang S, Lin H Z, Hwang J, Chacko V P, Diehl A M: Hepatic hyperplasia in noncirrhotic fatty livers: is obesity-related hepatic steatosis a premalignant condition? Cancer Res 61:5016-5023, 2001). Thus, the degrees of ERK phosphorylation in the livers of mice fed a high-fat diet (HFD mice) and ob/ob mice were examined using the Western blotting method. As the high-fat diet (HFD) mice, C57Bl/6N mice fed a high-fat diet (32% safflower oil, 33.1% casein, 17.6% sucrose, 5.6% cellulose) for 4 weeks since the age of 5 weeks were used. The ob/ob mice (Charles River Laboratories Japan, Inc.) used were of 8 weeks of age. As a result, increased phosphorylation of ERK was observed also in the livers of the HFD mice, as was in the ob/ob mice (FIG. 4a).

Similarly, db/db mice which exhibit insulin resistance and develop pancreatic islet hypertrophy (C57Bl/6 KSJ background) were infected with the Ad expressing the dominant-negative mutant MEK1 gene at 5×10⁸ PFU/body (Ueyama T, Kawashima S, Sakoda T, Rikitake Y, Ishida T, Kawai M, Yamashita T, Ishido S, Hotta H, Yokoyama M: Requirement of activation of the extracellular signal-regulated kinase cascade in myocardial cell hypertrophy. J Mol Cell Cardiol 32:947-960, 2000). The hepatic ERK phosphorylation decreased at 7 days after the viral administration (FIG. 4b). The pancreatic insulin content significantly decreased compared with that of the LacZ mice (FIG. 4c).

Thus, activation of the ERK pathway in the liver was involved also in physiological proliferation of pancreatic β cells and pancreatic islet hypertrophy.

### Example 4

===Effect of CAM Gene Transfer to Insulin-Deficient Model Mice===

Loss of β cells leads to absolute or relative insulin deficiency. The CAM virus was infected into the liver of mice suffering from diabetes at 1.5×10⁸ PFU/body, and the effect was examined. The mice used in the following experiments were the STZ mouse, a type 1 diabetes model mouse; the AKITA mouse, which has progressively decreasing insulin secretion; and the C57BL/KSJ-db/db mouse, a type 2 diabetes model mice which develop insulin resistance associated with obesity.

(1) STZ Mice in which β Cells were Destroyed by Administration of STZ

(i) Test Animals

The STZ mice were generated by administering STZ (Sigma) at 150 mg/kg to 8-week-old C57Bl/6N mice. Only those showing elevated fasting blood glucose levels of 250 mg/dl or above at 16 days after the administration were used.

(ii) Effect of Gene Transfer

The mice whose fasting blood glucose levels were elevated to 250 mg/dl or greater at 16 days after STZ administration were selected and were given by CAM viral injection into the liver (STZ-CAM mice) in the same manner as described above.

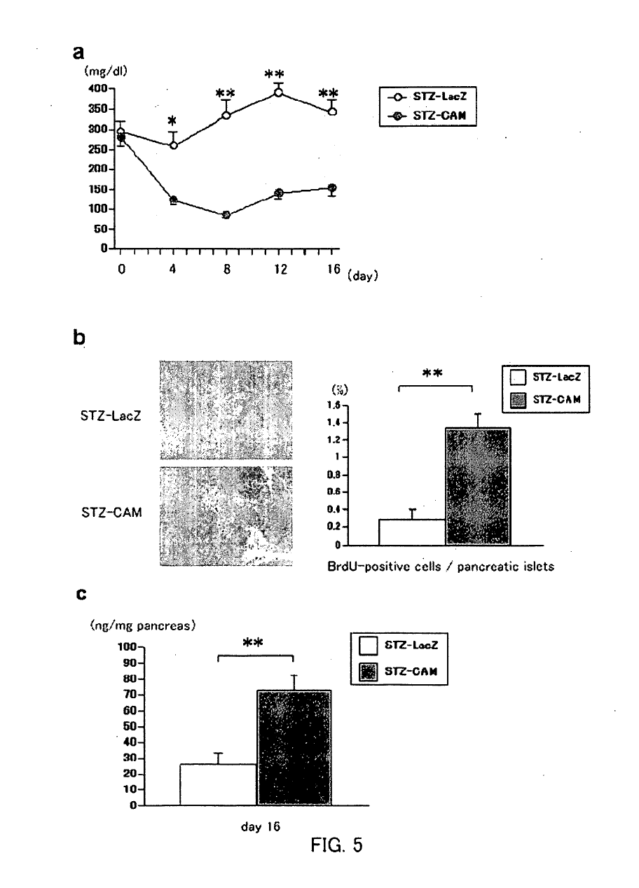

A significant decrease was observed in the blood glucose levels of the STZ-CAM mice at 4 days after the viral administration, compared with those of the STZ which were given by the LacZ gene transfer (STZ-LacZ mouse), and the levels returned to normal values. Further, the decrease was sustained up to 16 days after the viral administration (FIG. 5a). In addition, a significant increase was found in the BrdU positive-cells within the pancreatic islets of the STZ-CAM mice at 3 days after the viral administration (FIG. 5b). Also, a significant increase was observed in the pancreatic insulin content of the STZ-CAM mice at 16 days after the viral administration, compared with that of the STZ-LacZ mice (FIG. 5c).

(2) AKITA Mice in which β Cells are Lost by Endoplasmic Reticulum Stress (ER Stress)

(i) Test Animals

Five-week-old AKITA male mice (Kyudo Co., Ltd.) were used in the following experiment.

(ii) Effect of Gene Transfer

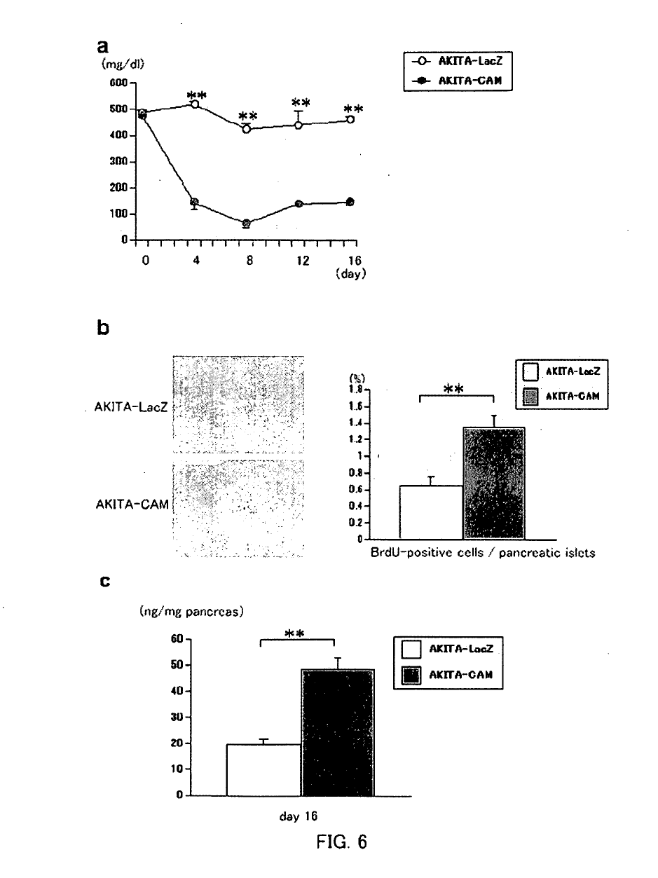

The CAM and LacZ gene transfers (the AKITA-CAM mice and AKITA-LacZ mice, respectively) were also carried out to the AKITA mice. A significant decrease was observed in the blood glucose levels of the AKITA-CAM mice at 4 days after the viral administration, compared with those of the AKITA-LacZ mice, and the decrease was sustained up to 16 days after the viral administration (FIG. 6a). In addition, a significant increase was observed in the BrdU positive-cells within the pancreatic islets of the AKITA-CAM mice at 3 days after the viral administration (FIG. 6b). Also, a significant increase was observed in the pancreatic insulin content of the AKITA-CAM mice at 16 days after the viral administration, compared with that of the STZ-LacZ mice (FIG. 6c).

(3) db/db Mice which Exhibit Marked Leptin Resistance and Insulin Resistance Due to Leptin Receptor Deficiency

(i) Test Animals

Eight-week-old db/db male (C57Bl/6 KSJ background) mice (Charles River Laboratories Japan, Inc.) were used.

(ii) Effect of Gene Transfer

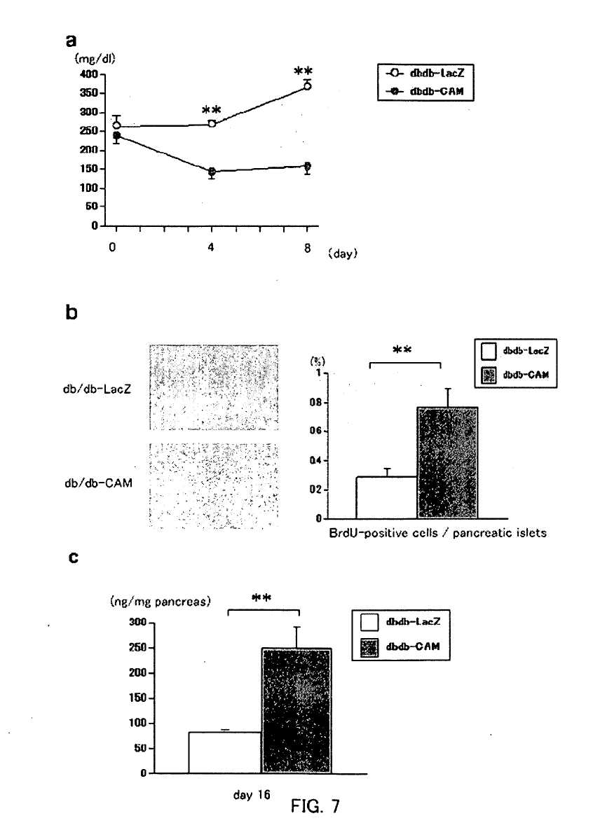

The CAM and LacZ gene transfers (db/db-CAM mice and db/db-LacZ mice, respectively) were also performed to the db/db mice in the same way. A significant decrease was observed in the blood glucose levels of the db/db-CAM mice at 4 days after the viral administration, compared with those of the db/db-LacZ mice, and the decrease was sustained up to 16 days after the viral administration (FIG. 7a). In addition, a significant increase was observed in the BrdU positive-cells within the pancreatic islets of the db/db-CAM mice at 3 days after the viral administration (FIG. 7b). Also, a significant increase was observed in the pancreatic insulin content of the db/db-CAM mice at 16 days after the viral administration, compared with that of the STZ-LacZ mice (FIG. 7c).

(4) Conclusion

As these findings indicate, enhancement of the function of ERK proteins in the liver deceases the blood glucose levels of the mice which have developed diabetes by loss of β cells, causes the β cell proliferation, and increases the amount of insulin secretion. Enhancement of ERK proteins in the liver is, therefore, effective in prevention/treatment of diabetes.

1. A method for promoting proliferation of a pancreatic β cell in a vertebrate, comprising the step of enhancing the function of an ERK protein in the liver of the vertebrate.

2. The method for promoting proliferation of a pancreatic β cell of claim 1, wherein an endogenous MEK protein is activated in the liver of the vertebrate.

3. The method for promoting proliferation of a pancreatic β cell of claim 1, wherein an active-form of MEK protein or a substance causing expression of an active-form of ERK protein is introduced into the liver of the vertebrate.

4. The method for promoting proliferation of a pancreatic β cell of claim 3, wherein an expression vector expressing a gene encoding the active-form of MEK protein is introduced into the liver of the vertebrate.

5. A method for increasing blood insulin concentration in a vertebrate, comprising enhancing the function of an ERK protein in the liver of the vertebrate.

6. The method for increasing blood insulin concentration of claim 5, wherein an endogenous MEK protein is activated in the liver of the vertebrate.

7. The method for increasing blood insulin concentration of claim 5, wherein an active-form of MEK protein or a substance causing expression of an active-form of MEK protein is introduced into the liver of the vertebrate.

8. The method for increasing blood insulin concentration of claim 7, wherein an expression vector expressing a gene encoding the active-form of MEK protein is introduced into the liver of the vertebrate.

9. A method for decreasing blood glucose level in a vertebrate, comprising enhancing the function of an ERK protein in the liver of the vertebrate.

10. The method for decreasing blood glucose level of claim 9, wherein an endogenous MEK protein is activated in the liver of the vertebrate.

11. The method for decreasing blood glucose level of claim 9, wherein an active-form of MEK protein or a substance causing expression of an active-form of MEK protein is introduced into the liver of the vertebrate.

12. The method for decreasing blood glucose level of claim 11, wherein an expression vector expressing a gene encoding the active-form of MEK protein is introduced into the liver of the vertebrate.

13. A method for preventing and/or treating diabetes in a vertebrate, comprising enhancing the function of an ERK protein in the liver of the vertebrate.

14. The method for preventing and/or treating diabetes of claim 13, wherein an endogenous MEK proteins is activated in the liver of the vertebrate.

15. The method for preventing and/or treating diabetes of claim 13, wherein an active-form of MEK protein or a substance causing expression of an active-form of MEK protein is introduced into the liver of the vertebrate.

16. The method for preventing and/or treating diabetes of claim 15, wherein an expression vector expressing a gene encoding the active-form of MEK protein is introduced into the liver of the vertebrate.

17. The method for preventing and/or treating diabetes of claim 13, wherein the diabetes is type 1 diabetes or type 2 diabetes.

18. The method for preventing and/or treating diabetes of claim 13, wherein the diabetes is either diabetes resulting from loss of pancreatic β cells or diabetes resulting from pancreatic β cell disorder.

19. A method for stimulating a vagus nerve controlling the pancreas in a vertebrate, comprising enhancing the function of an ERK protein in the liver of the vertebrate.

20. The method for stimulating a vagus nerve of claim 19, wherein an endogenous MEK protein is activated in the liver of the vertebrate.

21. The method for stimulating a vagus nerve of claim 19, wherein an active-form of MEK protein or a substance causing expression of an active-form of MEK protein is introduced into the liver of the vertebrate.

22. The method for stimulating a vagus nerve of claim 21, wherein an expression vector expressing a gene encoding the active-form of MEK protein is introduced into the liver of the vertebrate.
